(19) United States
(12) Patent Application Publication
(10) Pub. No.: US 2025/0146999 A1
(43) Pub. Date: May 8, 2025

(54) Simple shear assembly for three-dimensional geotechnical testing equipment

(71) Applicants: Tsinghua University, Beijing (CN); Zhengzhou University, Zhengzhou City (CN); China Construction Seventh Engineering Division Corp., LTD., Zhengzhou City (CN)

(72) Inventors: Dakuo Feng, Zhengzhou City (CN); Jianmin Zhang, Beijing (CN); Qingshan Wang, Zhengzhou City (CN)

(21) Appl. No.: 19/013,237

(22) Filed: Jan. 8, 2025

(30) Foreign Application Priority Data
Jul. 15, 2024 (CN) 202410942396.5

(51) Int. Cl. G01N 33/24; G01N 3/24

(52) U.S. Cl. CPC G01N 33/24; G01N 3/24

(57) ABSTRACT

A simple shear assembly includes: a base plate detachably mounted on a loading platform, and a stacked ring group opposite to the base plate; wherein the stacked ring group comprises: a plurality of stacked rings capable of sliding relative to each other; each of the stacked rings comprises a mounting frame and a holding frame provided within the mounting frame; the mounting frame comprises a support portion and a protective portion fixed to a top part of the support portion; the protective portion comprises a limiting groove and a rolling surface provided in the limiting groove; a support portion of an above adjacent stacked ring is rolled and supported on the rolling surface, in such a manner that all the stacked rings freely stagger with each other to perform interface simple shear. The protective portion can prevent the stacked rings from overturning.

### CROSS REFERENCE OF RELATED APPLICATION

The present invention claims priority under 35 U.S.C. 119 (a-d) to CN 202410942396.5, filed Jul. 15, 2024.

### BACKGROUND OF THE PRESENT INVENTION

### Field of Invention

The present invention relates to a technical field of geotechnical shear test equipment, and more particularly to a simple shear assembly for three-dimensional geotechnical testing equipment.

### Description of Related Arts

Shear test is an important means to examine the behavior of the soil-structure interface, and to reveal the deformation law of the interface. Among test equipment, direct shear and simple shear test equipment are most widely used in interface shear test.

The direct shear equipment is simple in structure and operation. During sample preparation, the upper shear box holds the soil sample and the lower box is poured with a structure such as concrete. The lower shear box is usually fixed during the test, and the upper shear box is either pushed or pulled to produce horizontal displacement. During the test, the vertical force could remain constant, and the horizontal shear force and horizontal displacement will be measured. Direct shear has simple test principles, but the failure position is artificially determined. During the shear process, stress and strain distribution may be nonuniform, the stress concentrates at the boundary, it is hard to distinguish between sliding displacement of contact surface and tangential displacement caused by soil deformation, etc.

Simple shear equipment can overcome some of the shortcomings of the direct shear equipment, such as the ability to distinguish between sliding displacement and tangential displacement caused by soil deformation and the more uniform distribution of stress and strain. In the shear test, besides a sliding surface, a shear zone with a certain thickness can be formed, which is more in line with the actual situation.

Although the simple shear equipment has improved some shortcomings of the direct shear equipment such as the nonuniform stress and strain distribution and the concentrated stress at the boundary, it has some problems in sample preparation as well as tangential displacement measurement due to large stacked ring horizontal displacement which leads to the stacked ring group overturning.

### SUMMARY OF THE PRESENT INVENTION

In view of the above, an object of the present invention is to provide a simple shear assembly for three-dimensional geotechnical testing equipment, so as to solve the problems of conventional simple shear equipment in sample preparation as well as tangential displacement measurement due to large stacked ring horizontal displacement which leads to the stacked ring group overturning.

Accordingly, in order to accomplish the above objects, the present invention provides a simple shear assembly for three-dimensional geotechnical testing equipment, comprising:
a base plate detachably mounted on a Y-direction loading platform, and a stacked ring group opposite to the base plate;
wherein the stacked ring group comprises: a plurality of stacked rings capable of sliding relative to each other;
wherein each of the stacked rings comprises a mounting frame and a holding frame provided within the mounting frame;
wherein the mounting frame comprises a support portion and a protective portion fixed to a top part of the support portion; the protective portion comprises a limiting groove and a rolling surface provided in the limiting groove; a support portion of an above adjacent stacked ring is rolled and supported on the rolling surface, in such a manner that all the stacked rings freely stagger with each other to perform interface simple shear.

Beneficial effect: the stacked ring group of the present invention comprises a plurality of stacked rings sliding relative to each other, wherein each of the stacked rings comprises a mounting frame and a holding frame provided within the mounting frame; wherein the mounting frame comprises a support portion and a protective portion fixed to a top part of the support portion; the protective portion comprises a limiting groove and a rolling surface provided in the limiting groove; a support portion of an above adjacent stacked ring is rolled and supported on the rolling surface, in such a manner that all the stacked rings freely stagger with each other to perform interface simple shear; the protective portion can limit the upper stacked rings when the upper stacked rings move horizontally with respect to the lower stacked rings in a large scale, so as to avoid overturning of the stacked ring group due to the upper stacked rings move horizontally with respect to the lower stacked rings in a large scale.

Preferably, the mounting frame is annular; the support portion and the protective portion are detachably connected; the support portion comprises an annular plate and a support section fixed to the annular plate; the support section is annular, and cross sections of the annular plate and the support section form an inverted T-shaped structure.

Beneficial effect: the annular mounting frame is easier to process and produce, and the stacked rings can move more freely relative to each other; the detachable connection of the support portion and the protective portion can facilitate the assembly and disassembly of the mounting frame relative to the upper and lower adjacent stacked rings.

Preferably, the protective portion is annular, and the limiting groove of the protective portion is adapted to the support portion; the protective portion further comprises an annular bottom plate, an inner retaining ring fixed to an internal side of the annular bottom plate, an outer retaining ring fixed to an external side of the annular bottom plate, an inner restriction plate fixed on a top of the inner retaining ring, and an outer restriction plate fixed on a top of the outer retaining ring; wherein a distance between the inner retaining ring and the outer retaining ring is larger than a cross-sectional width of the annular plate for the annular plate to move horizontally and freely in the limiting groove; after installing adjacent stacked rings, an inner retaining ring and an inner restriction plate of a lower stacked ring together restrict an annular plate of a support portion of an upper stacked ring to an inner side of the lower stacked ring, and an outer retaining ring and an outer restriction plate of the lower stacked ring together restrict the annular plate of the support portion of the above stacked ring to an outer side of the lower stacked ring.

Beneficial effect: the protective portion not only enables the stacked rings to move freely with respect to each other, but also limits the movement of the stacked rings in the horizontal direction; if the stacked ring group has a tendency to overturn, the inner restriction plate and the outer restriction plate can also limit the stacked rings in the vertical direction, further preventing the stacked ring group from overturning; furthermore, the protective portion has a simpler structure, and is easy to setup, process and produce.

Preferably, a mounting seat is fixed in the limiting groove, which is equipped with multiple balls; the balls form an annular rolling surface for rolling support of the annular plate of the support portion.

Beneficial effect: the mounting seat can facilitate the mounting limit of the balls, so that the balls form the annular rolling surface for rolling support of the support portion.

A height of the holding frame in a vertical direction is less than a height of the mounting frame in the vertical direction; a bottom surface of the holding frame is located above a bottom surface of the mounting frame, in such a manner that when the support portion of the upper stacked ring presses against a rolling surface of the lower stacked ring, a bottom surface of a holding frame of the upper stacked ring comes into contact with a top surface of a holding frame of the lower stacked ring.

Beneficial effect: when the support portion of the upper stacked ring presses against a rolling surface of the lower stacked ring, a bottom surface of a holding frame of the upper stacked ring comes into contact with a top surface of a holding frame of the lower stacked ring, which enables the mounting frame of the upper stacked ring to freely stagger with the mounting frame of the lower stacked ring when the holding frames of the upper and lower stacked rings move relative to each other.

The mounting frame is fixed to the holding frame through an annular connecting portion.

Beneficial effect: the connecting portion leaves a certain distance between the mounting frame and the holding frame in a radial direction of the stacked rings, preventing the mounting frames from interfering with the holding frames when the stacked rings stagger with each other.

Each of the stacked rings is fixed with two positioning lugs, and positioning holes are drilled on the positioning lugs; the two positioning lugs are arranged at 180° on each of the stacking rings; the positioning holes are threaded with positioning screws, and the positioning screws are detachably connected to the base plate for positioning of the stacked rings.

Beneficial effect: the positioning lugs and the positioning screws can easily restrict the stacked rings in the radial direction.

The base plate of the simple shear assembly is detachably connected to a mounting plate, and a carrier plate is fixed on the base plate; a transition disk is fixed on the carrier plate, and a simple shear bottom box is set on the transition disk and moves up and down with respect to the transition disk; a structure plate is set above the stacked ring group, which forms a second interface with an uppermost holding frame of the stacked ring group; the simple shear bottom box forms a first interface with a in lowermost holding frame of the stacked ring group; an elastic telescope structure is set between the simple shear bottom box and the carrier plate.

Beneficial effect: the first interface is formed between the lowermost holding frame of the stacked ring group and the simple shear bottom box; furthermore, an elastic telescope structure is set between the simple shear bottom box and the carrier plate, which enables the simple shear bottom box and the stacked ring group to move up and down freely, so that the stacked ring group and the structure plate are basically bonded together during shearing, thereby effectively respond to the shear transformation of the interface.

Preferably, the elastic telescopic structure comprises: a holder fixed on the carrier plate, a guide cylinder fixed to the holder and extending in a vertical direction, a guide column set in the guide cylinder and fixed to the holder, and a telescopic cylinder fixed to the simple shear bottom box; wherein the telescopic cylinder is set inside the guide cylinder; a guide end is provided at a bottom end of the telescopic cylinder for sliding with the guide cylinder; a through-hole is drilled in the telescopic cylinder for the guide column to extend into, and the guide column is suitable for guiding with an internal peripheral surface of the telescopic cylinder; a spring is provided on the guide column, wherein one end of the spring is fixed to the guide end, and the other end is fixed to the holder.

Beneficial effect: the elastic telescopic structure not only enables the simple shear bottom box and the stacked ring group to move up and down freely, but also guides the simple shear bottom box during moving, preventing the simple shear bottom box from tilting sideways during moving.

Preferably, a bottom positioning part corresponding to the positioning lugs is provided on the simple shear bottom box; transition holes corresponding to the positioning holes of the positioning lugs are drilled on the bottom positioning part, and the positioning screws pass through the positioning holes and the transition holes in turn and then are threaded with the carrier plate.

Beneficial effect: the simple shear bottom box and stacked ring group are uniformly restricted before the shearing process.

### BRIEF DESCRIPTION OF THE DRAWINGS

FIG. 1 is a structural view of three-dimensional geotechnical testing equipment containing a simple shear assembly of the present invention;

FIG. 2 is a top view of the three-dimensional geotechnical testing equipment in FIG. 1 after removing a top plate;

FIG. 3 is a structural view of the simple shear assembly for the three-dimensional geotechnical testing equipment according to the present invention;

FIG. 4 is a sketch view of a stacked ring group in FIG. 3;

FIG. 5 is a partial enlarge view of part A in FIG. 4;

FIG. 6 is a sketch view of a stacked ring in FIG. 3;

FIG. 7 is a top view of the stacked ring in FIG. 6;

FIG. 8 is a sketch view of an elastic telescoping structure in FIG. 3;

FIG. 9 is a sectional view of a structure plate in FIG. 3;

FIG. 10 is a partially enlarged view of part B in FIG. 9; and

FIG. 11 is a structural view of the simple shear assembly together with a simple shear top box according to the present invention.

Element reference: 1: base; 2: column; 3: top plate; 4: moving plate; 5: first kinematic pair; 6: mounting plate; 7: second kinematic pair; 8: second driving structure; 9: telescopic structure; 10: pressurized plate; 11: Y-direction laser displacement sensor; 12: X-direction laser displacement sensor; 13: first driving structure; 14: second guide rail; 15: base plate; 16: carrying plate; 17: stacked ring group; 20: positioning screw; 21: mounting frame; 22: connecting portion; 23: holding frame; 24: positioning lug; 25: positioning hole; 26: support portion; 27: limiting groove; 28: mounting seat; 29: ball; 30: annular plate; 31: support ring; 32: inner retaining ring; 33: inner restriction plate; 34: outer retaining ring; 35: outer restriction plate; 36: first guide rail; 42: transition disk; 43: simple shear bottom box; 44: structure plate; 45: elastic telescopic structure; 46: holder; 47: guide cylinder; 48: telescopic cylinder; 49: guide end; 50: guide column; 51: spring; 52: external ring plate; 53: core plate; 54: first ring groove; 55: second ring groove; 56: first lug; 57: second lug; 58: connecting bolt; 59: rubber film; 60: simple shear top box.

### DETAILED DESCRIPTION OF THE PREFERRED EMBODIMENT

A simple shear assembly for three-dimensional geotechnical testing equipment according to the present invention will be further illustrated in conjunction with accompanying drawings and preferred embodiments.

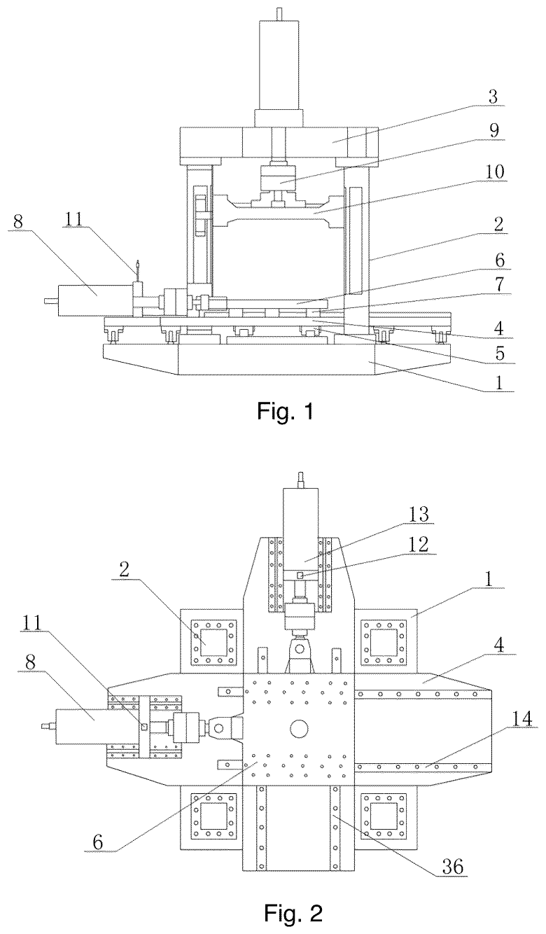

In order to facilitate the description of the simple shear assembly for three-dimensional geotechnical testing equipment. The structure of the three-dimensional geotechnical testing equipment will be introduced first. Referring to FIGS. 1 and 2, the three-dimensional geotechnical testing equipment comprises: a base 1, four columns 2 fixed and symmetrically arranged on the base 1, and a top plate 3 fixed to top ends of the columns 2. The base 1, the column 2 and the top plate 3 together form a frame structure of the testing equipment of the present invention. In the embodiment, the columns have a rectangular cross-section.

An X-direction loading platform is provided on the base 1, comprising a horizontally disposed moving plate 4, a first kinematic pair 5 disposed between the base 1 and the moving plate 4, and a first driving structure 13 on the base 1 that drives the moving plate 4 to move back and forth. In the embodiment, the first kinematic pair 5 comprises a first guide rail 36 fixed on the base 1 and a first slider fixed on the moving plate 4 that cooperates with the first guide rail 36. The first rail 36 extends along an X-direction. In the embodiment, the X-direction is a front-back direction. The first driving structure 13 is a first hydraulic cylinder capable of telescoping in the X-direction, and a telescoping end of the first hydraulic cylinder is fixed to the moving plate 4, so that when the first hydraulic cylinder telescopes in the X-direction, it can drive the moving plate 4 to move horizontally in the X-direction.

A Y-direction loading platform is provided on the moving plate 4, comprising a horizontally disposed mounting plate 6, a second kinematic pair 7 disposed between the mounting plate 6 and the moving plate 4, and a second driving structure 8 on the moving plate 4 that drives the mounting plate 6 to move in a left-right direction. In the embodiment, the second kinematic pair 7 comprises a second guide rail 14 fixed on the moving plate 4 and a second slider fixed on the mounting plate 6 that cooperates with the second guide rail 14. The second guide rail 14 extends along a Y direction. In the embodiment, the Y-direction is the left-right direction. The second driving structure 8 is a second hydraulic cylinder capable of telescoping in a Y-direction, and a telescoping end of the second hydraulic cylinder is fixed to the mounting plate 6, so that when the second hydraulic cylinder telescopes in the Y-direction, it can drive the mounting plate 6 to move horizontally in the Y-direction.

The simple shear assembly is provided on the mounting plate 6. In the embodiment, a vertical loading structure is provided on the top plate 3, comprising a telescopic structure 9 that can telescope in an up-down direction, and a pressurized plate 10 fixed on a bottom end of the telescopic structure 9, which can press against the simple shear assembly after moving downwards. In the embodiment, the telescopic structure 9 is a third hydraulic cylinder capable of telescoping in the vertical direction, and a telescopic end of the third hydraulic cylinder is fixed on the pressurized plate 10, so that when the third hydraulic cylinder telescopes in the vertical direction, it can drive the pressurized plate 10 to move in the up-down direction to apply loads to the simple shear assembly in the same direction.

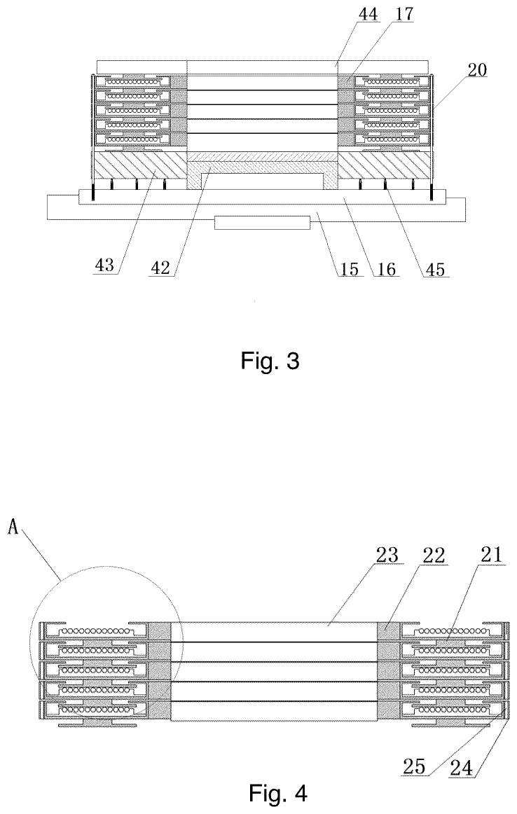

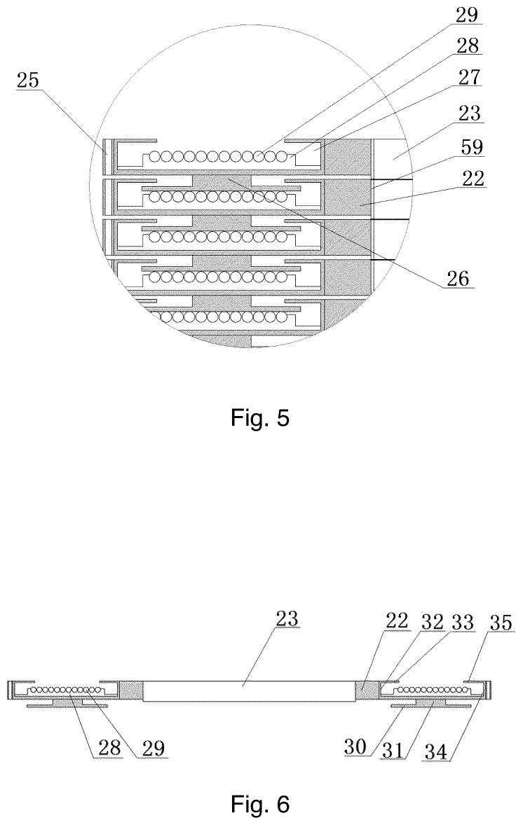

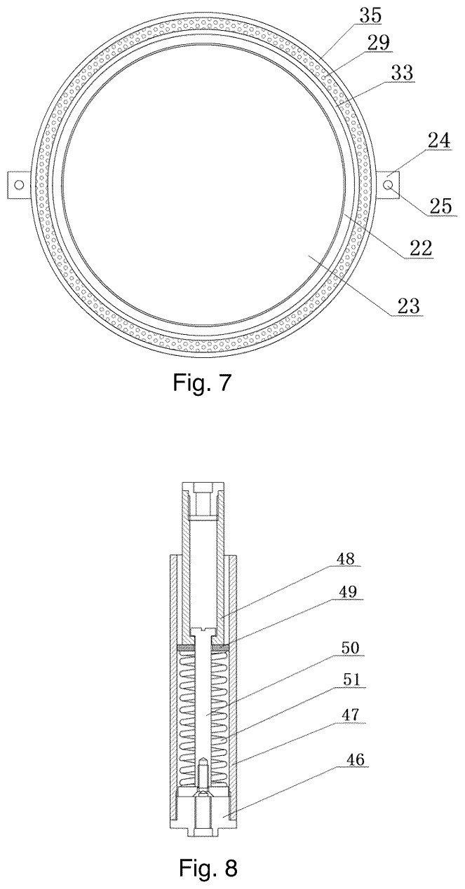

Referring to FIGS. 3-8, the simple shear assembly comprises a base plate 15 detachably connected to the mounting plate 6, and a stacked ring group provided on the base plate 15. Specifically, a carrier plate 16 is fixed on the base plate 15; a transition disk 42 is fixed on the carrier plate 16, and a simple shear bottom box 43 is set on the transition disk 42 and moves up and down with respect to the transition disk 42; a structure plate 44 is set above the stacked ring group 17; the simple shear bottom box 43 forms a first interface with a lowermost holding frame of the stacked ring group 17. In the embodiment, the simple shear bottom box is internally layered with a latex film to prevent soil particles from leaking into stacked ring gaps during shearing. The holding frames of the stacked ring group hold the same soil form, thereby facilitating observation of soil body shear deformation between stacked rings of the stacked ring group 17. A structure plate 44 is provided on the stacked ring group, and a vertical load applied by the vertical loading structure is applied to the structure plate 44, so that the applied vertical load can be more evenly distributed on the simple shear assembly through the transferring effect of the structure plate 44.

An elastic telescopic structure 45 is set between the simple shear bottom box 43 and the carrier plate, comprising: a holder 46 fixed on the carrier plate, a guide cylinder 47 fixed to the holder 46 and extending in the vertical direction, a guide column 50 set in the guide cylinder 47 and fixed to the holder 46, and a telescopic cylinder 48 fixed to the simple shear bottom box 43; wherein the telescopic cylinder 48 is set inside the guide cylinder 47; a guide end 49 is provided at a bottom end of the telescopic cylinder 48 for sliding with the guide cylinder 47; a through-hole is drilled in the telescopic cylinder 48 for the guide column 50 to extend into, and the guide column 50 is suitable for guiding with an internal peripheral surface of the telescopic cylinder 48; a spring 51 is provided on the guide column 50, wherein one end of the spring 51 is fixed to the guide end 49, and the other end is fixed to the holder 46. The elastic telescopic structure 45 not only enables the simple shear bottom box 43 and the stacked ring group to move up and down freely, but also guides the simple shear bottom box 43 during moving, preventing the simple shear bottom box 43 from tilting sideways during moving.

The stacked ring group 17 is provided on the simple shear bottom box. In the embodiment, the stacked ring group 17 comprises a plurality of stacked rings moving vertically and sliding horizontally relative to each other, wherein the stacked rings re circular, which ensures force balance of the soil body in all directions during three-dimensional loading, and can perform interface shear tests under any loading path. Each of the stacked rings comprises a mounting frame 21 and a holding frame 23 provided within the mounting frame 21. The holding frame 23 is filled with soil for sample making. The mounting frame 21 comprises a support portion 26 and a protective portion fixed to a top part of the support portion 26; the protective portion comprises a limiting groove 27 and a rolling surface provided in the limiting groove 27; a support portion 26 of an above adjacent stacked ring is rolled and supported on the rolling surface, in such a manner that all the stacked rings freely stagger with each other to perform interface simple shear. Specifically, both the mounting frame 21 and the holding frame 23 are annular to facilitate the assembly between the upper and lower stacked rings. In the embodiment, the support portion 26 is detachably connected to the protective portion. The support portion 26 comprises an annular plate 30 and a support ring 31 fixed to the annular plate 30. The support ring 31 is annular so that the annular plate 30 and the support ring 31 together form the annular support portion 26. A cross section of the support portion 26 is an inverted T-shaped structure. In other embodiments, the stacking ring may be square, in which case the mounting frame square and the holding frame should also be square.

The protective portion is annular, and the limiting groove 27 of the protective portion is adapted to the support portion 26; the protective portion further comprises an annular bottom plate, an inner retaining ring 32 fixed to an internal side of the annular bottom plate, an outer retaining ring 34 fixed to an external side of the annular bottom plate, an inner restriction plate 33 fixed on a top of the inner retaining ring 32, and an outer restriction plate 35 fixed on a top of the outer retaining ring 34; wherein a distance between the inner retaining ring 32 and the outer retaining ring 34 is larger than a cross-sectional width of the annular plate 30 for the annular plate 30 to move horizontally and freely in the limiting groove 27; after installing adjacent stacked rings, an inner retaining ring 32 and an inner restriction plate 33 of a lower stacked ring together restrict an annular plate of a support portion 26 of an upper stacked ring to an inner side of the lower stacked ring, and an outer retaining ring 34 and an outer restriction plate 35 of the lower stacked ring together restrict the annular plate 30 of the support portion 26 of the above stacked ring to an outer side of the lower stacked ring.

In the embodiment, a support portion is provided underneath the structure plate 44, and the support portion of the structure plate 44 is supported on the rolling surface of the uppermost stacked ring of the stacked ring group 17.

In the embodiment, a mounting seat 28 is fixed in the limiting groove 27, which is equipped with multiple balls 29; the balls 29 form an annular rolling surface for rolling support of the annular plate 30 of the support portion 26. In the embodiment, an inner diameter of the rolling surface formed by the balls 29 is smaller than an inner diameter of the annular plate 30 of the support portion 26, and an outer diameter of the rolling surface formed by the balls 29 is larger than the outer diameter of the annular plate 30 of the support portion 26, in such a manner that the rolling surface can provide stable rolling support for the support portion 26 when the support portion 26 moves relative to the rolling surface.

A height of the holding frame 23 in a vertical direction is less than a height of the mounting frame 21 in the vertical direction; a bottom surface of the holding frame 23 is located above a bottom surface of the mounting frame 21, in such a manner that when the support portion 26 of the upper stacked ring is placed into the limiting groove 27 of the protective portion of the lower stacked ring and presses against a rolling surface, a bottom surface of a holding frame 23 of the upper stacked ring comes into contact with a top surface of a holding frame 23 of the lower stacked ring. In the embodiment, the support portion 26 of the upper stacked ring forms an interface with the rolling surface of the lower stacked ring, and a distance between adjacent contact surfaces is equal to the height of the holding frame 23 in the vertical direction.

The mounting frame 21 is fixed to the holding frame 23 through an annular connecting portion 22. The connecting portion 22 leaves a certain distance between the mounting frame 21 and the holding frame 23 in a radial direction of the stacked rings, preventing the mounting frames 21 from interfering with the holding frames 23 when the stacked rings stagger with each other.

In the embodiment, each of the stacked rings is fixed with two positioning lugs 24, and positioning holes are drilled on the positioning lugs 24; the two positioning lugs 24 are arranged at 180° on each of the stacking rings; the positioning holes 25 are threaded with positioning screws 20, and the positioning screws 20 are detachably connected to the base plate. Specifically, a bottom end of the positioning screw 20 is threaded with a threaded hole on the carrier plate 16 for positioning the stacked rings under a combined effect of the positioning screw 20 and the positioning lug 24, so that the adjacent stacked rings are kept in overlapping form before loading of the stacked ring group 17.

An internal peripheral surface of the simple shear bottom box 43 is flush with an internal peripheral surface of the annular connecting portion 22, and an external peripheral surface of the simple shear bottom box 43 is flush with an external peripheral surface of the stacked rings. A bottom positioning part corresponding to the positioning lugs 24 is provided on the simple shear bottom box 43; transition holes corresponding to the positioning holes 25 of the positioning lugs 24 are drilled on the bottom positioning part, and the positioning screws 20 pass through the positioning holes 25 and the transition holes in turn and then are threaded with the carrier plate 16, thereby positioning the stacked rings.

During a simple shear test, each stacked ring is first assembled and lubricant is applied to the internal peripheral surface of the holding frame 23 of the stacked ring. concrete is poured in the holding frame 23 and vibrated on a vibrating plate. The transition disk 42 is fixed on the carrier plate, and then the simple shear bottom box 43 is set on the carrier plate. The elastic telescopic structure 45 is provided between the simple shear bottom box 43 and the carrier plate. After the concrete is completely solidified, the stacked rings are stacked on the simple shear bottom box 43 one by one, so that the lowermost stacked ring is in contact with the simple shear bottom box 43. Then the positioning screw 20 downwardly passes through the positioning holes 25 and is then threaded with the screw hole on the carrier plate for temporary fixation of the stacked rings relative to the carrier plate. The structure plate 44 is set on the stacked ring group, and then a vertical loading mechanism is adjusted so that a telescopic structure 9 of the vertical loading mechanism extends out. Now the pressurized plate 10 presses against the structure plate 44 and applies a downward load to the stacked ring group 17, and the X-direction and Y-direction loading platforms are adjusted in turn. At this time, the adjacent stacked rings stagger with each other, so as to shear the concrete in the holding frames 23 of the adjacent stacked rings, thereby facilitating the observation of microscopic changes of the soil body between the adjacent stacked rings during shear displacement.

In the embodiment, in order to prevent the soil from entering the gaps between the stacked rings, a rubber film 59 is provided inside the holding frame 23 of the stacked rings. An upper end of the rubber film 59 is fixed with a top stacked ring of the stacked ring group 17, while a lower end of the rubber file 59 is fixed with a bottom stacked ring of the stacked ring group 17, in such a manner that when the soil is placed in the holding frame, the rubber film isolates the soil from the structure of the stacked rings, so as to avoid the soil from entering the gaps between the stacked rings.

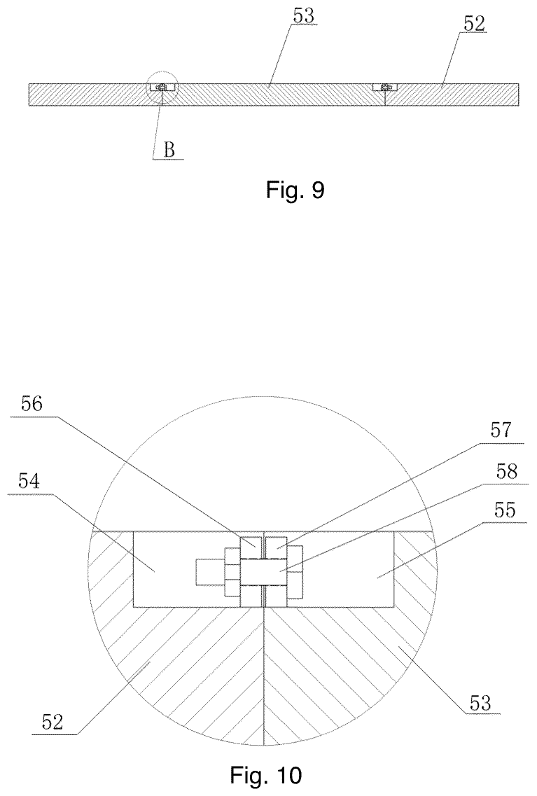

In the embodiment, referring to FIGS. 9 and 10, the structure plate 44 comprises a detachable external ring plate 52, a core plate 53 disposed inside the external ring plate 52, a first ring groove 54 provided on an internal peripheral surface at a top portion of the external ring plate 52, a second ring groove 55 provided on an external peripheral surface at a top portion of the core plate 53, a plurality of first lugs 56 uniformly disposed in the first ring groove 54, second lugs 57 disposed in the second ring groove 55 in correspondence with the first lugs 56, and connecting bolts 58 connecting the first lugs 56 and second lugs 57. When the external ring plate 52 and the core plate 53 are fixed together, it is possible for the pressurized plate 10 loaded in the vertical direction to act directly on the structure plate formed by the external ring plate 52 and the core plate 53, so as to perform an interfacial shear test of the soil and the structure. When only the core plate 53 is used, it is possible for the pressurized plate 10 loaded in the vertical direction to act directly on the core plate 53 to perform a direct shear test of the soil.

According to the present invention, the measurement of the relative dislocation of the stacked ring group is performed by laser displacement sensors. White marking points are provided in two orthogonal tangential directions (X-direction and Y-direction) of each stacked ring in the stacked ring group. At the same time, an X-direction laser displacement sensor 12 and a Y-direction laser displacement sensor 11 are provided corresponding to the three-dimensional geotechnical testing equipment. During testing, relative positions of the stacked ring marking points are measured through controlling the two laser displacement sensors in coordination with the marking points. In the embodiment, measurement of the relative dislocation between stacked rings by means of the laser displacement sensors are commonly known in the art, and will not be discussed herein.

The stacked ring group of the present invention is suitable not only for three-dimensional loading tests, but also for straight shear tests of soil.

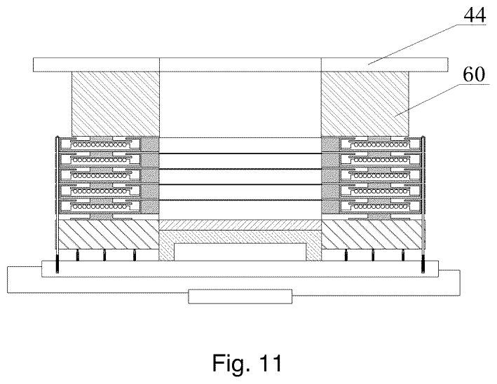

Referring to FIG. 11, when the positioning screw 20 is threaded through the positioning hole of the stacked ring group, each stacked ring in the stacked ring group is fixed by the positioning screw. With a simple shear top box 60 arranged on the stacked ring group, the whole device can be transformed into a straight shear instrument assembly. That is to say, the simple shear bottom box of the stacked ring group 17 can cooperate with the simple shear top box 60 for sending the soil body into the above-mentioned container, thereby performing straight shear test of soil.

In the present invention, each of the stacked rings comprises a mounting frame 21 and a holding frame 23 provided within the mounting frame 21. The mounting frame 21 comprises a support portion 26 and a protective portion fixed to a top part of the support portion 26. The protective portion comprises a limiting groove 27 and a rolling surface provided in the limiting groove 27. A support portion 26 of an above adjacent stacked ring is rolled and supported on the rolling surface, in such a manner that all the stacked rings freely stagger with each other to perform interface simple shear. The protective portion can limit the upper stacked ring when the upper stacked ring has a large horizontal movement relative to the lower stacked ring, avoiding the overturning of the stacked ring group 17 caused by a large horizontal displacement of the upper stacked ring relative to the lower stacked ring. When the stacked ring group 17 has a tendency to overturn, the inner restriction plate 33 and the outer restriction plate 35 can also limit the stacked rings in the vertical direction, further preventing the stacked ring group 17 from overturning.

In the above embodiments, the mounting frame is annular. In other embodiments, the mounting frame may also be rectangular.

In the above embodiment, the support portion and the protective portion are detachable. In other embodiments, the support portion and the protective portion may also be integrated; at this time, the inner restriction plate of the protective portion is detachably connected to the inner retaining ring, and the outer restriction plate is detachably connected to the outer retaining ring.

In the above embodiments, a mount seat is arranged in the limiting groove, which is equipped with multiple balls. The balls form an annular rolling surface for rolling support of the annular plate of the support portion. In other embodiments, the rolling surface may also be formed by a support roller.

In the above embodiments, the mounting frame is fixed to the holding frame through an annular connecting portion. In other embodiments, the annular connecting portion can be omitted, which means the mounting frame is directly fixed to the holding frame.

What is claimed is:

1. A simple shear assembly for three-dimensional geotechnical testing equipment, comprising:
a base plate detachably mounted on a Y-direction loading platform, and a stacked ring group opposite to the base plate;
wherein the stacked ring group comprises: a plurality of stacked rings capable of sliding relative to each other;
wherein each of the stacked rings comprises a mounting frame and a holding frame provided within the mounting frame;
wherein the mounting frame comprises a support portion and a protective portion fixed to a top part of the support portion; the protective portion comprises a limiting groove and a rolling surface provided in the limiting groove; a support portion of an above adjacent stacked ring is rolled and supported on the rolling surface, in such a manner that all the stacked rings freely stagger with each other to perform interface simple shear.

2. The simple shear assembly, as recited in claim 1, wherein the mounting frame is annular; the support portion and the protective portion are detachably connected; the support portion comprises an annular plate and a support section fixed to the annular plate; the support section is annular, and cross sections of the annular plate and the support section form an inverted T-shaped structure.

3. The simple shear assembly, as recited in claim 2, wherein the protective portion is annular, and the limiting groove of the protective portion is adapted to the support portion; the protective portion further comprises an annular bottom plate, an inner retaining ring fixed to an internal side of the annular bottom plate, an outer retaining ring fixed to an external side of the annular bottom plate, an inner restriction plate fixed on a top of the inner retaining ring, and an outer restriction plate fixed on a top of the outer retaining ring; wherein a distance between the inner retaining ring and the outer retaining ring is larger than a cross-sectional width of the annular plate for the annular plate to move horizontally and freely in the limiting groove; after installing adjacent stacked rings, an inner retaining ring and an inner restriction plate of a lower stacked ring together restrict an annular plate of a support portion of an upper stacked ring to an inner side of the lower stacked ring, and an outer retaining ring and an outer restriction plate of the lower stacked ring together restrict the annular plate of the support portion of the above stacked ring to an outer side of the lower stacked ring.

4. The simple shear assembly, as recited in claim 3, wherein a mounting seat is fixed in the limiting groove, which is equipped with multiple balls; the balls form an annular rolling surface for rolling support of the annular plate of the support portion.

5. The simple shear assembly, as recited in claim 1, wherein a height of the holding frame in a vertical direction is less than a height of the mounting frame in the vertical direction; a bottom surface of the holding frame is located above a bottom surface of the mounting frame, in such a manner that when a support portion of an upper stacked ring presses against a rolling surface of a lower stacked ring, a bottom surface of a holding frame of the upper stacked ring comes into contact with a top surface of a holding frame of the lower stacked ring.

6. The simple shear assembly, as recited in claim 4, wherein a height of the holding frame in a vertical direction is less than a height of the mounting frame in the vertical direction; a bottom surface of the holding frame is located above a bottom surface of the mounting frame, in such a manner that when the support portion of the upper stacked ring presses against a rolling surface of the lower stacked ring, a bottom surface of a holding frame of the upper stacked ring comes into contact with a top surface of a holding frame of the lower stacked ring.

7. The simple shear assembly, as recited in claim 1, wherein the mounting frame is fixed to the holding frame through an annular connecting portion.

8. The simple shear assembly, as recited in claim 4, wherein the mounting frame is fixed to the holding frame through an annular connecting portion.

9. The simple shear assembly, as recited in claim 2, wherein each of the stacked rings is fixed with two positioning lugs, and positioning holes are drilled on the positioning lugs; the two positioning lugs are arranged at 180° on each of the stacking rings; the positioning holes are threaded with positioning screws, and the positioning screws are detachably connected to the base plate for positioning of the stacked rings.

10. The simple shear assembly, as recited in claim 4, wherein each of the stacked rings is fixed with two positioning lugs, and positioning holes are drilled on the positioning lugs; the two positioning lugs are arranged at 180° on each of the stacking rings; the positioning holes are threaded with positioning screws, and the positioning screws are detachably connected to the base plate for positioning of the stacked rings.

11. The simple shear assembly, as recited in claim 9, wherein the base plate of the simple shear assembly is detachably connected to a mounting plate, and a carrier plate is fixed on the base plate; a transition disk is fixed on the carrier plate, and a simple shear bottom box is set on the transition disk and moves up and down with respect to the transition disk; a structure plate is set above the stacked ring group; the simple shear bottom box forms a first interface with a lowermost holding frame of the stacked ring group; an elastic telescopic structure is set between the simple shear bottom box and the carrier plate.

12. The simple shear assembly, as recited in claim 10, wherein the base plate of the simple shear assembly is detachably connected to a mounting plate, and a carrier plate is fixed on the base plate; a transition disk is fixed on the carrier plate, and a simple shear bottom box is set on the transition disk and moves up and down with respect to the transition disk; a structure plate is set above the stacked ring group; the simple shear bottom box forms a first interface with a lowermost holding frame of the stacked ring group; an elastic telescope structure is set between the simple shear bottom box and the carrier plate.

13. The simple shear assembly, as recited in claim 11, wherein the elastic telescopic structure comprises: a holder fixed on the carrier plate, a guide cylinder fixed to the holder and extending in a vertical direction, a guide column set in the guide cylinder and fixed to the holder, and a telescopic cylinder fixed to the simple shear bottom box; wherein the telescopic cylinder is set inside the guide cylinder; a guide end is provided at a bottom end of the telescopic cylinder for sliding with the guide cylinder; a through-hole is drilled in the telescopic cylinder for the guide column to extend into, and the guide column is suitable for guiding with an internal peripheral surface of the telescopic cylinder; a spring is provided on the guide column, wherein one end of the spring is fixed to the guide end, and the other end is fixed to the holder.

14. The simple shear assembly, as recited in claim 12, wherein the elastic telescopic structure comprises: a holder fixed on the carrier plate, a guide cylinder fixed to the holder and extending in a vertical direction, a guide column set in the guide cylinder and fixed to the holder, and a telescopic cylinder fixed to the simple shear bottom box; wherein the telescopic cylinder is set inside the guide cylinder; a guide end is provided at a bottom end of the telescopic cylinder for sliding with the guide cylinder; a through-hole is drilled in the telescopic cylinder for the guide column to extend into, and the guide column is suitable for guiding with an internal peripheral surface of the telescopic cylinder; a spring is provided on the guide column, wherein one end of the spring is fixed to the guide end, and the other end is fixed to the holder.

15. The simple shear assembly, as recited in claim 13, wherein a bottom positioning part corresponding to the positioning lugs is provided on the simple shear bottom box; transition holes corresponding to the positioning holes of the positioning lugs are drilled on the bottom positioning part, and the positioning screws pass through the positioning holes and the transition holes in turn and then are threaded with the carrier plate.

16. The simple shear assembly, as recited in claim 14, wherein a bottom positioning part corresponding to the positioning lugs is provided on the simple shear bottom box; transition holes corresponding to the positioning holes of the positioning lugs are drilled on the bottom positioning part, and the positioning screws pass through the positioning holes and the transition holes in turn and then are threaded with the carrier plate.
